(19) United States
(12) Patent Application Publication
(10) Pub. No.: US 2015/0080483 A1
(43) Pub. Date: Mar. 19, 2015

(54) POWER STATION-BASED METHANATION SYSTEM

(71) Applicant: Siemens Aktiengesellschaft, Munich (DE)

(72) Inventors: Marc Hanebuth, Nurnberg (DE); Uwe Lenk, Zwickau (DE); Nicolas Vortmeyer, Erlangen (DE)

(73) Assignee: Siemens Aktiengesellschaft, Munich (DE)

(21) Appl. No.: 14/390,990

(22) PCT Filed: Mar. 22, 2013
(86) PCT No.: PCT/EP2013/056065

§ 371 Date: Oct. 6, 2014

(30) Foreign Application Priority Data
Apr. 10, 2012 (EP) 12163588.2

(51) Int. Cl. C07C 1/04; C07C 1/12; B01J 7/00

(52) U.S. Cl. CPC C07C 1/0485; B01J 7/00; C07C 1/12
USPC 518/704; 422/162

(57) ABSTRACT

A power station-based methanation system which has a fossil fuel-fired power station together with an electrolysis unit and a methanation reactor is provided. The power station and the electrolysis unit are configured for supplying the methanation reactor with starting materials for a methanation reaction and the electrolysis unit can be operated both in a charging state and in a discharging state, in which charging state the electrolysis unit supplies electric power and a chemical energy store is at the same time charged and in which discharging state the chemical energy store is discharged.

### CROSS REFERENCE TO RELATED APPLICATIONS

This application is the US National Stage of International Application No. PCT/EP2013/056065 filed Mar. 22, 2013, and claims the benefit thereof. The International Application claims the benefit of European Application No. EP12163588 filed Apr. 10, 2012. All of the applications are incorporated by reference herein in their entirety.

### FIELD OF INVENTION

The present invention relates to a power plant-based methanation system which, as well as a fossil-fired power plant, includes an electrolysis unit and a methanation reactor. The invention further relates to methods for operating such a methanation system.

### BACKGROUND OF INVENTION

In spite of a growing proportion of power generation methods based on renewable energy sources in overall power generation, coverage of the baseload in the public power supply grid also requires the operation of fossil-fired power plants. These especially convert the chemical energies present in coal, natural gas, mineral oil or other raw materials to thermal energy, in order thus to maintain a power generation process.

For technical and economic reasons, fossil-fired power plants are preferably operated continuously under full load, and so they are particularly suitable for covering the baseload in the public power supply grids. In addition, the public power supply grids are supplied at irregular intervals with power from power generation processes fed by renewable energy sources. This sometimes leads to significant fluctuations in the provision of electrical power in the public power supply grids. In order to balance out these fluctuations, it is necessary to feed additional electrical power into the power supply grid in periods of significant power demand, and to remove electrical power from the public power supply grids in periods of oversupply thereof.

In order to utilize the temporary oversupply of electrical power in an economically viable manner, the excess power in electrical and in chemical form can be stored intermediately. For example, it is possible to utilize excess electrical power for synthetic production of methane (substitute natural gas, SNG). This synthetically produced methane can easily be produced from a few starting materials via catalytic methods known from the prior art. For example, methane, given suitable choice of the reaction conditions, can be prepared from the starting materials CO and hydrogen or CO₂ and hydrogen (Sabatier process). However, in order to conduct this production in an economically viable manner, it is necessary to undertake the synthesis reaction with maximum continuity and under full load.

A conventional method for producing methane or methanol is described, for example, in EP 2426236 A1. The basic idea of this method is based here on the intermittent supply of an electrolysis unit with electrical power for production of hydrogen, which is then converted together with carbon dioxide in a reactor unit to give methane and/or methanol. Through later combustion of this methane or the methanol in accordance with demand, it is possible, for instance, by means of a gas turbine process or a steam turbine process, to again provide electrical power. In this context, however, the method is found to be disadvantageous since the electrolysis unit can be operated only in periods of sufficient power supply, or, in an inefficient manner, the methane or methanol produced has to be combusted again for power provision, in order to operate the electrolysis unit. Thus, there is assurance neither of continuous operation of the electrolysis unit nor of sufficient economic viability in the methane or methanol production.

### SUMMARY OF INVENTION

It is consequently an object of the present invention to avoid the disadvantages from the prior art. More particularly, it is an object of the invention to enable essentially continuous production of synthetic methane. Moreover, the synthetic methane production is to be enabled in periods of oversupply of electrical power in the public power supply grids, and also in periods of elevated demand for electrical power therein. Thus, essentially continuous methane production is to be enabled, at least partly independently of the electrical power supply in the public power supply grids. At the same time, the energy is to be provided primarily through the temporary supply of excess power in the public power supply grids.

This object is achieved through a power plant-based methanation system and methods for operating such a methanation system according to the claims.

More particularly, this object is achieved by a power plant-based methanation system which, as well as a fossil-fired power plant, includes an electrolysis unit and a methanation reactor, wherein the power plant and the electrolysis unit are designed to supply the methanation reactor with starting materials for a methanation reaction, and wherein the electrolysis unit can be operated both in a charging state and in a discharging state, in which charging state the electrolysis unit is supplied with electrical power and, at the same time, a chemical energy storage means is charged, and in which discharging state the chemical energy storage means is discharged.

The object of the invention is also achieved by a method for operating an above-described methanation system which, to a fossil-fired power plant, includes an electrolysis unit and a methanation reactor, wherein the power plant and the electrolysis unit are designed to supply the methanation reactor with starting materials for a methanation reaction, wherein the electrolysis unit in a first step is supplied with water which, in a second step, is converted electrolytically or chemically to hydrogen, and wherein, in a third step, the hydrogen is mixed together with CO₂ from the power plant and the mixture of hydrogen and CO₂ is fed as starting material to the methanation reactor.

The object of the invention is also achieved by a method for operating an above-described methanation system which, as well as a fossil-fired power plant, includes an electrolysis unit and a methanation reactor, wherein the power plant and the electrolysis unit are designed to supply the methanation reactor with starting materials for a methanation reaction, wherein the electrolysis unit in a first step is supplied with a mixture of water and CO₂ from the power plant and said mixture, in a second step, is correspondingly converted electrolytically or chemically in the electrolysis unit to hydrogen and CO₂, and wherein, in a third step, the mixture of hydrogen together with the CO₂ is supplied as starting materials to the methanation reactor.

According to aspects of the invention, the power plant-based methanation system thus includes, as well as a methanation reactor for production of synthetic methane, a fossil-fired power plant which, as well as the provision of electrical power, can likewise provide starting materials for the methanation reaction. Such starting materials are especially CO and CO₂, which arise because of the combustion reaction in the fossil-fired power plant.

In addition, the inventive methanation system includes an electrolysis unit which can be operated both in a charging state and a discharging state. In the charging state, the electrolysis unit is supplied with electrical power either from the fossil-fired power plant or else preferably from the public power supply grids in the event of supply of excess power. At the same time, a chemical energy storage means is charged, and is discharged again during the discharging state, and thus supplies power to the production of the corresponding starting material required for methanation.

The charging state and discharging state may follow on immediately from one another.

The charging state should thus be regarded as a charging operation during which the electrolysis unit consumes electrical power and a chemical energy storage means is charged. The discharging state should be regarded as a discharging operation in which the charged chemical energy storage means is discharged again, in order to provide energy for the electrolysis.

The electrolysis unit can consequently be operated either with consumption of electrical power in the charging state or with release of chemical energy in the discharging state for electrolysis. This enables the provision of synthetically produced methane in periods of oversupply of electrical power in the public power supply grids, and also essentially continuous operation in the event of elevated power demand therefrom. Continuous operation in turn can make the intermediate storage of the starting materials superfluous, as a result of which the production can be effected in a very economically viable manner at high load on the plant.

It is envisaged in accordance with aspects of the invention that the electrolysis unit in the charging state stores at least a portion of the electrical power supplied as chemical energy in a chemical energy storage means. Accordingly, the chemical storage of the electrical power consumed during the charging state is effected not just indirectly in the form of the synthetically produced methane in the methanation reactor; instead, the chemical storage is additionally effected in the electrolysis unit itself. Given establishment of suitable reaction conditions, this chemical energy can be made utilizable again in suitable form, and allows the energization of downstream processes. More particularly, the provision of the intermediately stored chemical energy assures the operation of the methanation unit during the discharging state of the electrolysis unit.

According to aspects of the invention, the synthetic methane production is supplied directly or indirectly with starting materials from the fossil-fired power plant, and with starting materials from the electrolysis unit. Since both the fossil-fired power plant and the electrolysis unit can provide these starting materials essentially continuously, the methanation reaction in the methanation reactor can likewise proceed essentially continuously. Consequently, synthetic methane can be produced essentially irrespective of the power supply or of the power demand in the public power supply grids, without being restricted exclusively to supply with electrical power from the fossil-fired power plant.

In accordance with a form of the method, the electrolysis unit is supplied only with excess power from the public power supply grids during the charging state. Accordingly, it would be possible to completely dispense with supply of the electrolysis unit with electrical power from the fossil-fired power plant. Since, however, the power supply in the public power supply grids may be subject to sometimes significant fluctuations over the course of the day, it may also be necessary in practice to be able to draw electrical power from the fossil-fired power plant during the charging state of the electrolysis unit.

In the context of the present invention, the term “fossil-fired power plant” should be understood in its broadest meaning. More particularly, fossil-fired power plants also include combustion plants for refuse utilization.

In a first embodiment of the invention, the electrolysis unit, both in the charging state and in the discharging state, can produce at least one starting material through electrolysis. Consequently, the electrolysis unit is suitable for essentially continuous provision of starting materials for the methanation reaction. More particularly, the electrolysis unit, in a further development, is capable of producing molecular hydrogen during the charging state and also during the discharging state. This can be reacted with the CO₂ or CO, or a mixture of these two substances, which form as combustion products in the power plant process, in the methanation reactor under suitable reaction conditions to give synthetic methane.

In a further embodiment of the methanation system, the electrolysis unit needs to be supplied with an air stream for operation of a gas electrode or for removal of the oxygen formed during the charging state. More particularly, the electrolysis unit has a connection through which the air stream can be introduced into the electrolysis unit. In addition, the air stream is particularly suitable for removing heat from the electrolysis unit. In practice, the air stream may also take the form of a general gas stream.

In practice, the electrolysis unit may correspond in terms of its configuration to the battery described in WO 2011/070006 A1. This published specification is hereby explicitly incorporated into the present application by reference. The battery described therein, which corresponds essentially to the present electrolysis unit in terms of its construction, has numerous gas channels, by means of which oxygenous process gas is conducted to a cathode. The oxygenous process gas, in order to reduce potential hazard and also in order to increase economic viability, is atmospheric oxygen.

The oxygen present in the process gas is reduced during the discharging state and passes through the ion-conductive cathode. Because of the oxidation potentials that prevail, the reduced oxygen migrates further through a solid-state electrolyte to an anode at which the ionic oxygen releases its charge and joins together with molecular hydrogen to form water. The solid-state electrolyte here is advantageously suitable for anionic conductivity, but prevents electrical conduction of charge carriers. The solid-state electrolyte comprises, for example, a metal oxide, for instance zirconium oxide and/or cerium oxide, which has in turn been doped with a metal, for example scandium. Because of the doping, oxygen vacancies are produced in the metal oxide, and these allow anionic transport of reduced oxygen (i.e. double negatively charged oxygen atoms) or increase the stability of the electrolyte.

Because of the reducing agent present at the anode, for example molecular hydrogen, the anionic oxygen is converted to H₂O according to the following equation:

H₂+O²⁻→H₂O+2e⁻ (eq. 1)

The electrons released here can be tapped off at the anode and sent to an electrical load circuit.

In contrast to the above-described discharging state of the battery described in WO 2011/070006 A1, the discharge of the present electrolysis unit does not proceed with release of electrical power. Instead, in the electrolysis unit in the discharging state, the chemical energy stored in the chemical energy storage means is utilized to drive the production of a starting material required for the methanation.

For provision of a starting material for the methanation reaction, for example, gaseous water is introduced for this purpose into a support body including oxidizable material, preferably in the form of an elemental metal. The material may be in the form of powder or else in the form of porous compressed bodies. On the reaction of the gaseous water with the oxidizable material which serves as reducing agent for the water, elemental hydrogen is produced, for example, according to the following equation:

Me+H₂O→MeO+H₂ (eq. 2)

In this equation, Me represents an oxidizable material, especially a metal, and constitutes the chemical energy storage means in the electrolysis unit. This can be produced, i.e. regenerated again, through suitable reduction of the oxidized material during the charging state. On account of suitably selected electronegativities of the oxidizable material, the tendency of the gaseous water to react with the material in the support body is stronger than, for instance, with a metal of the anode material. As a result, the anode material is advantageously protected from corrosion. Further details with regard to the specific structure of the battery or of the electrolysis unit can be taken from the above-designated published specification.

In a further advantageous embodiment, the electrolysis unit comprises a metal and/or a metal oxide as chemical energy storage means which can be oxidized during the discharging state. The metal and/or the metal oxide thus enable the chemical energy stored therein to be released again in a suitable manner during the discharging state. The metal and/or metal oxide may preferably be from the group of lithium, manganese, iron, titanium and tungsten. Preferably, the metal and/or metal oxide is in the form of powder or porous compressed bodies. More particularly, such a metal and/or metal oxide enables a suitable reaction with gaseous water, as indicated above in equation 2. This releases molecular hydrogen, which can serve as starting material for the methanation reaction.

In a further embodiment of the inventive methanation system, the electrolysis unit comprises a metal oxide which can be reduced during the charging state. On completion of reduction, the metal oxide is in a relatively lower oxidation state, or in pure metallic form, and can thus provide a chemical energy storage means which, in the course of discharge, supplies the chemical energy for the chemical conversion in the electrolysis unit. The reduction of the metal oxide also releases oxygen, which can in turn be used as oxidizing agent again. In accordance with the battery described above, the structure of which may correspond to the present electrolysis unit, the oxygen encompassed by the metal oxide may be suitable for formation of molecular oxygen, the molecular oxygen being released to a certain degree as a by-product during the charging state. During the charging state, the metal oxide is thus reduced to the elemental metal or to a comparatively lower-valency metal oxide, which can in turn be available as a chemical energy storage means during the discharging state.

In accordance with a further embodiment of the methanation system, the electrolysis unit has an inlet which is designed to supply the electrolysis unit with water, especially with steam. The water/the steam is provided as process material or process gas which allows operation of the electrolysis unit in a charging or discharging state. Preferably, steam may also be provided during the discharging state as a transport material for elemental oxygen. Through reaction with a metal, for example, to give a metal oxide, the oxygen is chemically bound and consequently assures the chemical storage of electrical power. Equally, the water or the steam may serve to provide elemental oxygen during the charging state, and the hydrogen required for the methanation reaction can be provided through release of oxygen.

Water and steam are inexpensive here and are comparatively non-hazardous in relation to the handling thereof.

In a further embodiment of the invention, the electrolysis unit comprises a solid-state electrolyte which especially electrically insulates two electrical electrodes from one another, but has a predetermined ion conductivity, especially an anion conductivity. The solid-state electrolyte advantageously assures electrical insulation, which is the prerequisite for controlled electrical operation of the electrolysis unit. On account of the selective ion conductivity, it is possible to achieve a controlled discharging state and charging state. At the same time, the solid-state electrolyte prevents the mixing of process gases which interact, for example, during the charging or discharging state with one of the two electrical electrodes and/or both electrical electrodes.

In a further embodiment of the inventive methanation system, the electrolysis unit is suitable for operation at at least 500° C., especially at least 600° C. and preferably between 600° C. and 800° C. The high operating temperatures assure efficient charging and discharging operation, and consequently efficient provision of starting materials for the methanation reaction. In addition, the waste heat from the electrolysis unit may advantageously serve as waste heat, for instance, for preheating of the starting materials before they are introduced into the methanation reactor.

In accordance with another embodiment of the inventive methanation system, the power plant includes a CO₂ removal device which is designed to remove CO₂ from an offgas stream from the power plant and to provide gaseous CO₂ as starting material for the methanation reaction in the methanation reactor and/or for the electrolysis for the electrolysis unit. The CO₂ removal device consequently assures the processing, especially the selective processing, of the offgas stream from the power plant, in order to be able to provide the starting material which is converted to synthetic methane during the methanation reaction. Other contaminating substances are not selectively removed for utilization in the CO₂ removal device here, and consequently do not contribute significantly, if at all, to impurities in the methanation reactor. The selective removal of CO₂ increases the efficiency with which synthetic methane can be produced. In addition, it increases the purity of the synthetic methane produced in the methanation reactor. Moreover, the selective removal of CO₂ from the offgas stream from the power plant enables essentially quantitatively controlled supply of CO₂ to the methanation reactor.

In a further embodiment of the methanation system, a thermal bridge is also provided, and is designed to pass thermal energy from the methanation reactor to the electrolysis unit. The thermal bridge is especially intended for conducting positive and negative thermal energy, meaning that the heat conduction may be in either direction. Since the methanation reaction is typically strongly exothermic, the heat released can be used for preheating of the electrolysis unit. This heat is supplied to the electrolysis unit by means of the thermal bridge. Equally, the heat released in the methanation reaction is suitable for conditioning of the water introduced into the electrolysis unit, in order to evaporate it, for example. In practice, it is thus possible to utilize some or most of the heat obtained during the methanation reaction for operation of the electrolysis unit. It is likewise conceivable that the heat is utilized for preheating of process gas streams which are fed to the electrolysis unit. This utilization of heat consequently increases the overall efficiency of the methanation system in practice.

In addition, it simplifies heat management in the course of operation of the methanation system.

In a further embodiment of the inventive methanation system, a water recycling system is provided, which is designed to feed water which has been obtained after the methanation reaction back to the electrolysis unit or to the inlet which is designed at least to supply the electrolysis unit with water. The water recycling system thus reduces firstly excess water consumption, and secondly unwanted energy consumption for thermal conditioning of the water supplied to the electrolysis unit. Since the water obtained after the methanation reaction typically still has a large heat content, it requires relatively little energy to re-evaporate this water for use in the electrolysis unit. Recycling of water thus enables efficient management of mass and heat.

A first embodiment of the method of the invention provides for especially continuous operation of the electrolysis unit in either a charging state or a discharging state. The continuous operation of the electrolysis unit assures the continuous provision of starting materials for the methanation reaction. Consequently, it is unnecessary to store these starting materials prior to supply in the methanation reactor. The continuous operation of the electrolysis unit additionally enables a high economic efficiency of operation.

In a further development of this method, there may be alternation between the charging and discharging states. Such alternation of the charging and discharging states can achieve efficient continuous operation. In this case, the electrolysis unit is converted from the discharging state to a charging state especially when sufficient amounts of starting materials can no longer be provided for the methanation reaction during the energetically self-sufficient discharging state. Consequently, controlled alternation of the charging state and discharging state can achieve an advantageously high efficiency in the execution of the overall method.

In a further embodiment of the method of the invention, the starting materials are supplied to the methanation reactor as a mixture essentially in stoichiometric amounts. Preferably, the deviations from the required stoichiometric amounts are less than 20%, more preferably less than 10% and most preferably less than 5%. The methanation reactor is thus supplied essentially only with those amounts of starting materials which are actually converted in full during the methanation reaction. Consequently, the synthetic methane produced in the methanation reactor is contaminated by extraneous substances to a relatively small degree, such that complex gas separation is not required after production of the synthetic methane. If the conversion in the methanation reactor is complete, the synthetic methane withdrawn from the methanation reactor will merely be contaminated by water. However, the water, which is typically in vaporous form at the temperatures that prevail in the methanation reactor, can easily be condensed out and then fed back to the electrolysis unit, for example, via a suitable water recycling system.

In a further embodiment of the method of the invention, there is no intermediate storage of the starting materials after they have been withdrawn from the electrolysis unit. In practice, a flow system is thus present, which can do without the provision of intermediate stores. Such a flow system can be implemented in an advantageous manner especially when both the electrolysis unit and the methanation reactor can be operated in continuous flow.

This in turn assures a high process efficiency.

In a further embodiment of the method of the invention, the products which are withdrawn from the methanation reactor are not fed to a process for reconversion to power. It is likewise possible to feed the products directly to infrastructure for handling of natural gas as synthetic gas. Reconversion to power, which would result in thermal and electrical power loss, can consequently be avoided.

In a further modification of the method, the methanation reactor may be operated continuously. The continuous operation especially assures a high process efficiency and a desired continuous provision of synthetic methane.

The present invention is to be elucidated hereinafter with reference to working examples. It should be pointed out here that the narrowing of the invention in the working examples does not constitute any restriction in terms of the subject matter claimed in general. Moreover, the features elucidated in the working examples are claimed individually in themselves, and also in conjunction with other features.

Furthermore, it should be pointed out that the figures which follow are merely schematic diagrams. This, however, does not constitute a restriction in terms of the specific practice of the present invention.

### BRIEF DESCRIPTION OF THE DRAWINGS

The figures show the following:

FIG. 1 a schematic diagram of the methanation system of the invention in a first embodiment;

FIG. 2 a schematic diagram of the methanation system of the invention in a second embodiment;

FIG. 3 a schematic diagram of individual chemical reactions and processes during the operation of the electrolysis unit in a charging state;

FIG. 4 a schematic diagram of individual chemical reactions and processes during the operation of the electrolysis unit in a discharging state;

FIG. 5 a schematic flow diagram for illustration of a first embodiment of the method of the invention;

FIG. 6 a schematic flow diagram for illustration of a method of the invention in a second embodiment.

### DETAILED DESCRIPTION OF INVENTION

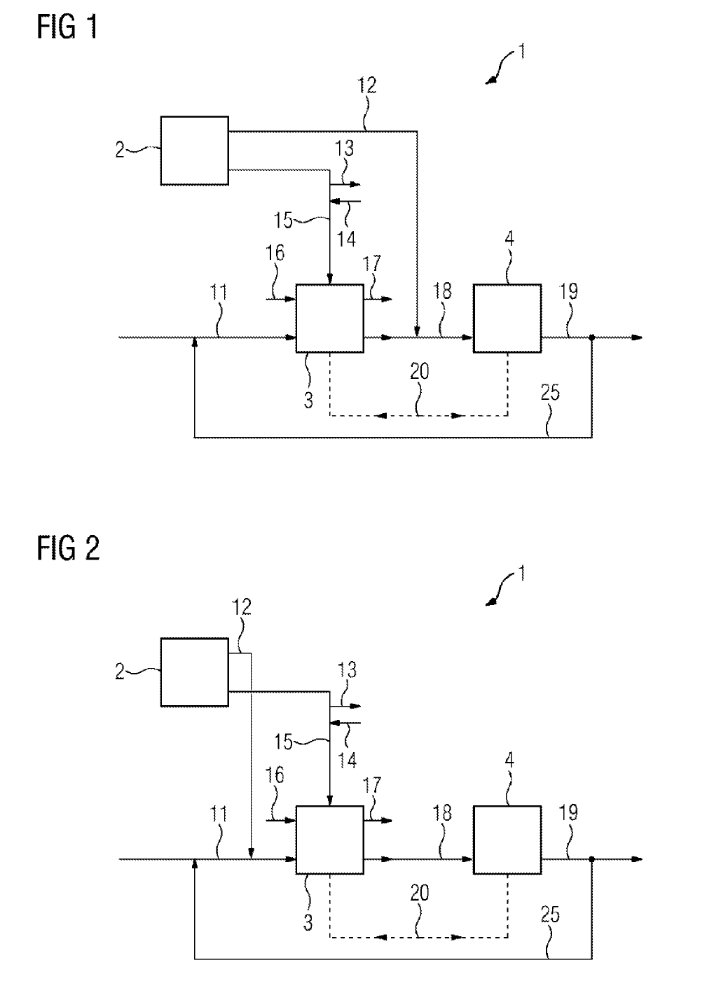

FIG. 1 shows a first embodiment of the inventive methanation system 1 which, as well as a fossil-fired power plant 2, also includes an electrolysis unit 3. Both the power plant 2 and the electrolysis unit 3 are intended for provision of starting materials 10 (not shown here) and for supply thereof to a methanation reactor 4 in which the starting materials 10 are converted chemically to synthetic methane in a suitable manner.

In this context, the fossil-fired power plant 2 provides gaseous CO₂ in a supply line 12. Preferably, the CO₂ provided has been removed in the power plant 2 by means of a CO₂ removal device, which is not shown in any detail, from an offgas stream from the power plant 2. For supply of electrical power to the electrolysis unit 3, a power supply line 15 is also provided, which allows supply of power to the electrolysis unit 3 from the fossil-fired power plant 2. Alternatively and/or additionally, electrical power can also be abstracted from the public power supply grids via a grid power abstraction line 14 and supplied to the electrolysis unit 3. It is likewise possible to supply electrical power from the power plant 2 via a grid power supply line 13 to the public power supply grids.

For the operation of the electrolysis unit 3 in a charging/discharging state, it may be necessary to supply air thereto via an air supply line 16. The gas supplied, after utilization of the electrolysis unit 3 as intended, or during the utilization of the electrolysis unit 3 as intended, is removed from the air outlet 17. After the withdrawal, this gas can be sent back, for example, to the ambient air. It serves firstly for suitable removal of heat and secondly for removal of the oxygen formed during the charging state.

Moreover, the operation of the electrolysis unit 3 requires the supply of a suitable process material which can be fed in through the inlet 11 of the electrolysis unit 3. In practice, this process material is water or steam, which is at least partly converted to hydrogen in the electrolysis unit 3 during the charging state and the discharging state. The starting material 10 produced by electrolysis or chemical reaction for the methanation reaction is fed by means of the transfer line 18 to the methanation reactor 4. If complete conversion of water is not achieved in the electrolysis unit 3, the unconverted amounts are at least partly also conveyed into the methanation reactor 4 together with the electrolytically produced hydrogen. According to the present embodiment, the supply line 12 for provision of CO₂ opens into the transfer line 18, such that the hydrogen can mix therein with the CO₂. This mixture is subsequently fed to the methanation reactor 4, in which the starting materials 10 are converted to synthetic methane. The synthetic methane thus produced is discharged from the methanation reactor by means of a product department 19.

During the methanation reaction, which proceeds with strong exothermicity, heat is released, which can be conducted in a suitable manner through the thermal bridge 20 to the electrolysis unit 3. The heat thus provided can serve, in the electrolysis unit 3, for preheating of the process gases used or for preheating of the water or for vaporization of the water. Because of this thermal conditioning, a much higher temperature level compared to the environment exists in the electrolysis unit 3. The temperature increase results in turn in a decrease in the electrolysis voltage, which for its part results in an improved cross section of action in the provision of the starting materials 10.

The product withdrawn from the product outlet 19, ideally a mixture of synthetic methane and water, requires a suitable removal of water from the product stream. This can be achieved, for example, through an advantageous condensation of the water present in the product stream, in which case the water can be fed via a water recycling system 25 back to the inlet 11 for provision to the electrolysis unit 3.

FIG. 2 shows a further embodiment of the inventive methanation system 1. It differs from the methanation system 1 shown in FIG. 1 merely in that the CO₂ provided by the supply line 12 is intended not for supply to the methanation reactor 4 into the transfer line 18, but for supply to the inlet 11, in order to be supplied to the electrolysis unit 3. Consequently, by means of the inlet 11 to the electrolysis unit 3, a mixture of CO₂ and water as process materials is fed in, in which case it is possible to correspondingly convert the two substances by electrolysis in the electrolysis unit 3. If CO₂ is converted to CO in the electrolysis unit 3, water, in accordance with the details given above, is converted to hydrogen. Both substances, hydrogen and CO, are fed as starting materials 10 from the electrolysis unit 3 via the transfer line 18 to the methanation reactor 4. In the methanation reactor 4, the two substances as starting materials are correspondingly converted to synthetic methane. If complete conversion of water and CO₂ is not achieved in the electrolysis unit 3, the unconverted amounts are also conveyed into the methanation reactor 4 together with the hydrogen and CO.

In this case, in the embodiment according to FIG. 2, and also in the embodiment according to FIG. 1, stoichiometric amounts of CO₂ can be supplied via the supply line 12. In other words, the amount of CO₂ supplied via the supply line 12 is just sufficient that the starting materials 10 supplied to the methanation reactor 4 can be converted stoichiometrically, i.e. essentially completely. In order to be able to suitably adjust the amounts of CO₂, it is possible here for setting means that are not shown in any detail, especially valves, to be provided.

In addition, the embodiment according to FIG. 1 and according to FIG. 2 illustrates that there is no provision of intermediate storage means in which the starting materials 10 would have to be stored intermediately before being supplied to the methanation reactor. Instead, both the fossil-fired power plant 2 and the electrolysis unit 3, and also the methanation reactor 4, are in essentially constant operation, such that synthetically produced methane can be withdrawn continuously from the product outlet 19.

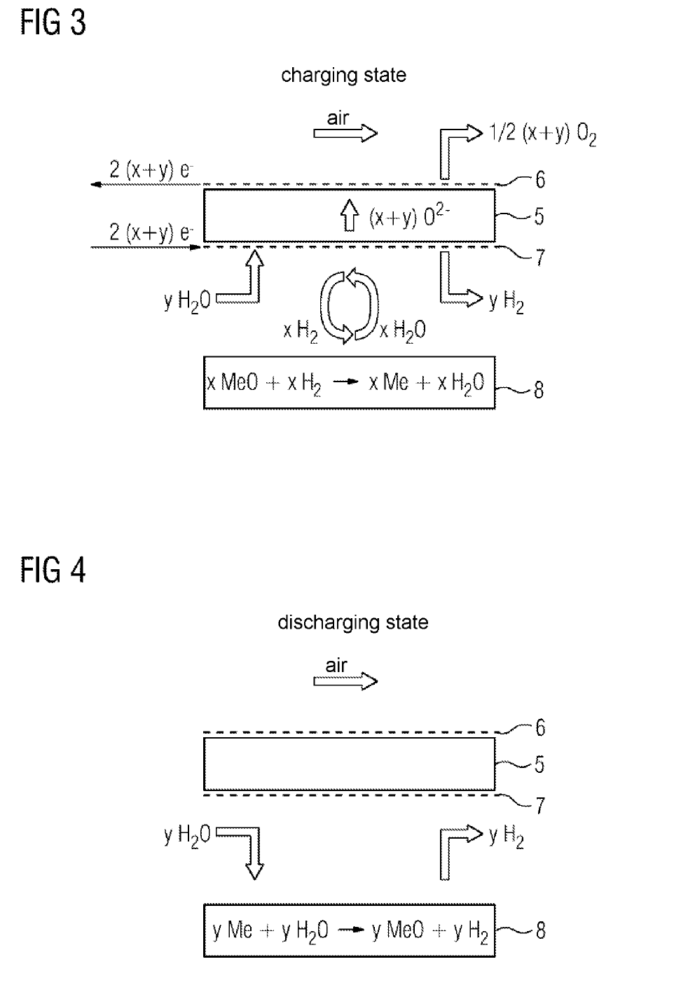

The chemical reactions and processes that proceed in the electrolysis unit 3 during a charging state are illustrated schematically in FIG. 3. In this figure, the electrolysis unit 3 comprises an arrangement composed of a first electrical electrode 6 and a second electrical electrode 7, both of which are electrically insulated from one another by a solid-state electrolyte 5. The first electrical electrode 6 is in direct contact here with air as process gas.

The first electrical electrode may, for example, comprise a substance having perovskite structure. It may have a layer thickness between 10 and 200 μm, preferably about 50 μm. The solid-state electrolyte 5 typically takes the form of a metal-doped metal oxide and has a layer thickness of typically between 20 and 100 μm, preferably 50 μm. The second electrode 7 may be configured as a metal-ceramic composite material, called a cermet, in which case advantageous metals may be lithium, manganese, iron, titanium, tungsten or nickel.

In practice, the second electrode 7 is in contact with gaseous water. In the same reaction space, there is likewise a metal oxide (MeO), which can be converted by molecular hydrogen to elemental metal (Me) and water. The metal serves here as a chemical energy storage means 8 during the discharging state shown in FIG. 4. During the charging state shown here, however, metal oxide is reduced back to the form suitable for chemical storage, namely the metal.

During the charging state, between the first electrode and the second electrode 7, there is an electrical potential which ensures an excess of electrons (e⁻) at the second electrode 7. Acceptance of two electrons from the second electrode 7 results in reduction of water to hydrogen (H₂), with simultaneous formation of a double negatively charged oxygen anion. This anion migrates from the second electrode 7 through the solid-state electrolyte 5 to the first electrode 6, where it releases its electrical charge again and reacts to give molecular oxygen. The molecular oxygen is removed together with the air which is in contact with the first electrode.

The hydrogen formed as a result of decomposition at the second electrode 7 reacts in turn with the metal oxide, forming metal in elemental form and water. The water formed in this reaction can in turn be reduced again at the second electrode 7 to hydrogen, again with formation of an oxygen anion which migrates to the first electrode 6 through the solid-state electrolyte 5.

The water molecule released from the reaction of the hydrogen with the metal oxide is consequently converted back to hydrogen at the second electrode 7. Accordingly, the reduction of the metal oxide to elemental metal sustains itself, namely in that each hydrogen molecule gives rise to one water molecule which leads in turn to formation of another hydrogen molecule at the second electrode 7. However, the situation is different for the water introduced from the outside, which is converted to hydrogen at the second electrode 7, but the hydrogen does not contribute again to reduction of the metal oxide. In practice, this hydrogen released in this way from the electrolysis unit 3 is discharged as starting material 10 for the methanation reaction in the methanation reactor 4.

If the electrolysis unit 3 is then operated in a discharging state rather than a charging state, the procedures shown schematically in FIG. 4 proceed. Unlike during the charging state, there is no electrical potential, which would drive the processes, between the first electrode 6 and the second electrode 7 during the discharging state. Instead, anion flow is prevented by the solid-state electrolyte 5. Anion migration through the solid-state electrolyte 5 is prevented, for example, via prevention of the drawing of electrical current via the two electrodes 6 and 7.

During the discharging state, water is introduced into the electrolysis unit 3, and reacts chemically with the metal, oxidizing the metal, to give hydrogen. Since this hydrogen does not react any further at the second electrode 7, it can be fed to the methanation reactor 4 as starting material 10 of the methanation reaction. The metal thus assumes the role of the chemical energy storage means 8, which provides the energy for the electrolysis of water to hydrogen as it proceeds.

According to the reactions that proceed, which are shown for the charging state in FIG. 3 and for the discharging state in FIG. 4, within the electrolysis unit, a proportion of water is always being converted to a proportion of hydrogen. The latter can serve as starting material 10 for the methanation reaction in the methanation reactor 4. In practice, hydrogen can thus be provided as starting material both during the charging state and during the discharging state, irrespective of the state of operation.

The reactions that proceed in the energy storage means 8 are described here by way of example for a divalent metal. However, this is not supposed to constitute a restriction. Instead, the principle can be applied to other suitable substances. In that case, the reaction equations change correspondingly.

As well as water as starting material for the electrolysis in the electrolysis unit 3, it is likewise possible to supply a mixture of CO₂ and water, in which case CO₂ is converted to CO and water to hydrogen. In this case, CO₂ is converted to CO through release of an oxygen atom at the second electrode 7.

In addition, it is entirely conceivable that the conversions in the electrolysis unit 3 do not proceed to completion, meaning that the conversion levels are not 100%.

Moreover, it may also be advisable to feed a proportion of the hydrogen produced by electrolysis from water back to the electrolysis unit 3, in order to prevent aging of the electrodes, especially of the electrode operated as cathode in the discharging state, or to reverse this aging.

FIG. 5 shows a schematic sequence of process steps, for illustration of a first embodiment of the method of the invention in a flow diagram. In this case, in a first process step, water is supplied to an electrolysis unit 3 (not identified here), in order that it is converted electrolytically or chemically to hydrogen in a second step therein. This conversion can be effected, as already elucidated in FIGS. 3 and 4, either during a discharging state or during a charging state of the electrolysis unit 3. In a third step, the hydrogen thus produced is mixed with CO₂ from a power plant 2 (not identified here). The CO₂ originates here preferably from a CO₂ removal device integrated into or connected downstream of the power plant 2. In a further step, both gases, hydrogen and CO₂, are fed as a mixture to the methanation reactor 4 (not identified here), in order to be converted therein to synthetic methane in a methanation reaction.

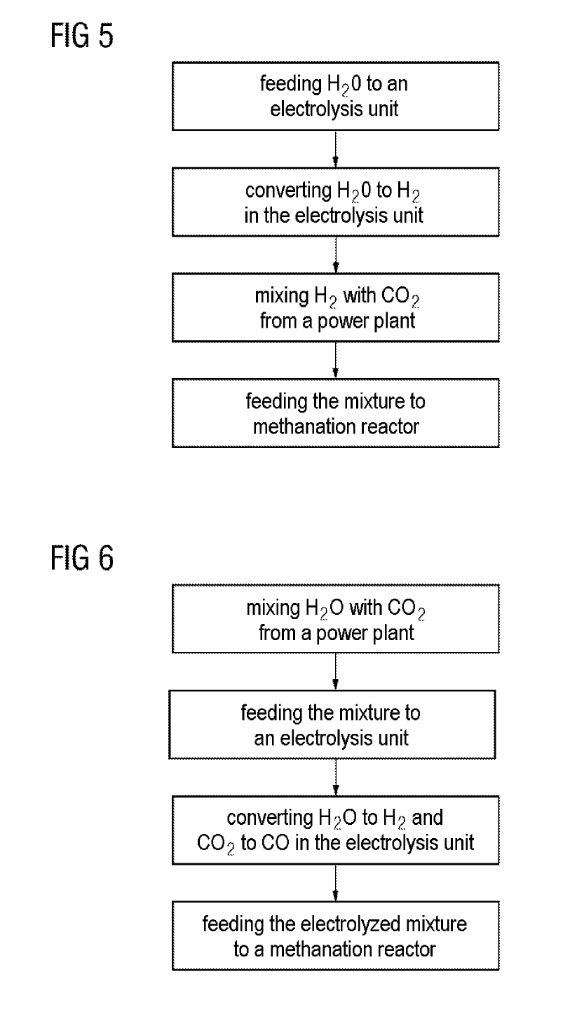

FIG. 6 shows a further embodiment of the method of the invention as a flow diagram. The process shown in FIG. 6 differs here from the process shown in FIG. 5 merely in that a mixture of water and CO₂ is fed to the electrolysis unit in a second step. In a third step, the electrolysis unit 3 electrolytically or chemically converts water to hydrogen and CO₂ to CO. In a fourth step, the mixture thus obtained is fed to a methanation reactor 4, such that the starting materials 10 present therein can be converted in a methanation reaction to synthetic methane.

1.-15. (canceled)

16. A power plant-based methanation system comprising:
a fossil-fired power plant, an electrolysis unit, and a methanation reactor,
wherein the power plant and the electrolysis unit are adapted to supply the methanation reactor with starting materials for a methanation reaction,
wherein the electrolysis unit is operable both in a charging state and in a discharging state, in which charging state the electrolysis unit is supplied with electrical power and, at the same time, a chemical energy storage means is charged in the electrolysis unit, and in which discharging state the chemical energy storage means is discharged and hence the production of the corresponding starting materials is supplied with power, and
wherein the electrolysis unit comprises a metal and/or a metal oxide as chemical energy storage means which can be oxidized during the discharging state.

17. The methanation system as claimed in claim 16,
wherein the electrolysis unit, both in the charging state and in the discharging state, can produce at least one starting material through electrolysis.

18. The methanation system as claimed in claim 16,
wherein the electrolysis unit comprises a metal oxide which can be reduced during the charging state.

19. The methanation system as claimed in claim 16,
wherein the electrolysis unit has an inlet which is adapted to supply the electrolysis unit with water.

20. The methanation system as claimed in claim 16,
wherein the electrolysis unit comprises a solid-state electrolyte which electrically insulates two electrical electrodes from one another, and has a predetermined ion conductivity.

21. The methanation system as claimed in claim 16,
wherein the electrolysis unit is adapted for operation at at least 500° C.

22. The methanation system as claimed in claim 16,
wherein the power plant includes a CO₂ removal device which is adapted to remove CO₂ from an offgas stream from the power plant and to provide gaseous CO₂ as starting material for the methanation reaction in the methanation reactor and/or for the electrolysis in the electrolysis unit.

23. The methanation system as claimed in claim 16,
further comprising a thermal bridge which is adapted to pass thermal energy from the methanation reactor to the electrolysis unit.

24. The methanation system as claimed in claim 16,
further comprising a water recycling system which is adapted to feed water which has been obtained after the methanation reaction back to the electrolysis unit or to the inlet which is adapted at least to supply the electrolysis unit with water.

25. A method for operating a methanation system described in claim 16 comprising a fossil-fired power plant, an electrolysis unit, and a methanation reactor, wherein the power plant and the electrolysis unit are adapted to supply the methanation reactor with starting materials for a methanation reaction, the method comprising:
in a first step, the electrolysis unit is supplied with water,
in a second step, the water is converted electrolytically or chemically to hydrogen, and
in a third step, the hydrogen is mixed together with CO₂ from the power plant and the mixture of hydrogen and CO₂ is fed as starting materials to the methanation reactor.

26. A method for operating a methanation system described in claim 16 comprising a fossil-fired power plant, an electrolysis unit, and a methanation reactor, wherein the power plant and the electrolysis unit are adapted to supply the methanation reactor with starting materials for a methanation reaction, the method comprising:
in a first step, the electrolysis unit is supplied with a mixture of water and CO₂ from the power plant,
in a second step, the mixture of water and CO₂ is correspondingly converted electrolytically or chemically in the electrolysis unit to hydrogen and CO, and
in a third step, the hydrogen and CO is supplied as starting materials to the methanation reactor.

27. The method as claimed in claim 25,
wherein the electrolysis unit is operated continuously, in either a charging state or a discharging state.

28. The method as claimed in claim 26,
wherein the electrolysis unit is operated continuously, in either a charging state or a discharging state.

29. The method as claimed in claim 25,
wherein the starting materials are supplied to the methanation reactor as a mixture in stoichiometric amounts.

30. The method as claimed in claim 26,
wherein the starting materials are supplied to the methanation reactor as a mixture in stoichiometric amounts.

31. The method as claimed in claim 25,
wherein the methanation reactor is operated continuously.

32. The method as claimed in claim 26,
wherein the methanation reactor is operated continuously.

33. The methanation system as claimed in claim 19,
wherein the electrolysis unit has an inlet which is adapted to supply the electrolysis unit with steam.

34. The methanation system as claimed in claim 20,
wherein the predetermined ion conductivity is an anion conductivity.

35. The methanation system as claimed in claim 21,
wherein the electrolysis unit is adapted for operation at at least 600° C.

36. The methanation system as claimed in claim 21,
wherein the electrolysis unit is adapted for operation between 600° C. and 800° C.
